(19) United States
(12) Patent Application Publication
(10) Pub. No.: US 2014/0309673 A1
(43) Pub. Date: Oct. 16, 2014

(54) DEVICES FOR REMOVING VESSEL OCCLUSIONS

(71) Applicant: Nathan John DACUYCUY, Irvine, CA (US)

(72) Inventors: Nathan John Dacuycuy, Irvine, CA (US); Jianlu Ma, Irvine, CA (US)

(21) Appl. No.: 14/357,547

(22) PCT Filed: Nov. 9, 2012
(86) PCT No.: PCT/US12/64533

§ 371 Date: May 9, 2014

Related U.S. Application Data
(60) Provisional application No. 61/607,915, filed on Mar. 7, 2012.
(60) Provisional application No. 61/558,550, filed on Nov. 11, 2011.

(51) Int. Cl. A61B 17/3207

(52) U.S. Cl. CPC A61B 17/320725
USPC 606/159

(57) ABSTRACT

Described herein are thrombectomy devices and methods of using same for capturing or encapsulating and removal of a blood or vessel.

### CROSS REFERENCE TO RELATED APPLICATIONS

This application claims the benefit of U.S. provisional patent application No. 61/558,550, filed Nov. 11, 2011 and U.S. provisional patent application No. 61/607,915, filed Mar. 7, 2012, the entire disclosures of which are incorporated herein by reference.

### FIELD

Described generally are devices and methods useful for emboli retrieval to treat, among other things, ischemic stroke.

### BACKGROUND

Currently, FDA-approved treatment options for an acute ischemic stroke include intravenous (IV) delivery of clot dissolving medicine, and mechanical thrombectomy devices.

For treatment using a clot dissolving medicine, the thrombolytic agent (Tissue Plasminogen Activator (t-PA)) is injected to the vasculature to dissolve the blood clot that is blocking blood flow to the neurovasculature. IV t-PA is currently limited in use because it must be used within a three hour window from the onset of a stroke and can result in an increased risk of bleeding.

For treatment using a mechanical thrombectomy device, the device physically captures an embolus or clot and removes it from the blocked vessel, thereby restoring blood flow. The major advantage of the mechanical thrombectomy device is it can expand the treatment windows from 3 hours to over 10 hours. Some existing mechanical thrombectomy devices used for increasing blood flow through an obstructed blood vessel include a filter trap designed and built to collect and remove emboli; a cork-screwed guidewire like device to retrieve an embolus; and a stent like device connected to a delivery wire to retrieve embolus.

Many disadvantages of the above mentioned mechanical thrombectomy devices exist. For example, for filter-like devices, filters tend to be cumbersome and difficult to deliver, deploy and a larger profile guide catheter may be needed to fully remove the embolus; difficulty exists in coordinating movement to a position precisely and predictably within a vessel; and the device can drift or twist within the vessel or not conform to the vessel wall making embolus removal difficult. Further, there is no immediate vascular recannalization during the procedure.

Cork-screwed guidewire like devices may only possess the ability to capture and remove embolus that are firm or independently held as one piece. Further, there is no immediate vascular recannalization during a cork-screwed guidewire procedure and the device is not capable of capturing small emboli that may break off from a large embolus.

The existing stent like mechanical thrombectomy devices may not be capable of capturing small emboli that may break off from the large embolus, and can lead to complications such as blockage of distal smaller vessels, vessel dissection, perforation and hemorrhage can arise as a result of over-manipulation in the vessel.

Other disadvantages of the above mentioned devices can also exist. For example, a device may capture an embolus, but then lose grasp and migrate/deposit it incidentally in another area of the neurovasculature, creating the potential for a new stroke in a different part of the neurovasculature. The devices may not be capable of capturing small embolus break-offs and preventing them from migrating to a more distal area of the neurovasculature. Also, the relative large device delivery profiles may prevent treatment of distal small diameter vessels.

Existing mechanical thrombectomy devices may also be built using two or more distinct pieces that require either joints and/or bonding between the delivery system and the treatment device. This connection generally results in a weakness in the device that can result in an unintentional separation of the two pieces, possibly leaving the treatment device in the body during embolus retrieval. Also, the treatment portion of mechanical thrombectomy devices (e.g., stent like devices) tend to be cut from tubing that is larger than the delivery system, thus making the treatment portion the limiting factor in terms of minimizing the compacted profile of the device, requiring larger access systems and greater delivery force to deliver the device. Other flaws in the current mechanical thrombectomy designs include poor visibility and/or radiopacity, lack of variation in the delivery portion to enhance and improve deliverability, and lack of coatings or modified surface textures on the treatment portion to enhance embolus affinity, etc.

None of the existing medical mechanical thrombectomy devices address the needs in the art.

### SUMMARY

Described herein generally are devices and methods useful for emboli retrieval and removal. Such devices and methods can treat, among other things, ischemic stroke. In one embodiment, medical devices are described that can be used as a mechanical thrombectomy device to retrieve and remove an obstruction responsible for a narrowing and/or blockage of vessel(s) in neurovasculature or cardiac vasculature to restore oxygenated blood flow or superoxygenated blood distal of the blockage while the obstruction is being cleared.

The device described can be formed of or from a single piece tubing giving the device a seamless transition from proximal delivery portion to distal therapeutic or treatment portion. The tubing can be any biocompatible material which exhibits super elastic or shape memory properties such as a nitinol super elastic material and/or a nitinol shape memory alloy material. This single piece device can remove any joints or bonding of a delivery wire with the treatment device thus eliminating physical weakness in the device and reducing unintentional breakage during device delivery/retrieval.

Also, the devices can include features which can be cut and/or implemented into device's proximal delivery portion such as a transition portion to achieve variable flexibility and profile for easy delivery and navigation. Non-limiting examples include spiral cuts, helix/coil configurations, etc. The flexibility of the proximal delivery portion can vary from proximal to distal. For example, the distal portion can be more flexible than proximal portion. Further, the device can achieve a smaller compacted profile, which reduces delivery and retrieval force allowing the physician to use smaller microcatheters for delivery to smaller vessels or more distal vasculature.

In one embodiment, thrombectomy devices are described comprising: a substantially cylindrical body formed from a single piece of tubing and including a proximal delivery portion, a transition portion, and a treatment portion, wherein the treatment portion includes one or more struts for encapsulating a luminal occlusion that may be potentially mobile, occlude blood flow, or both. The cylindrical body can be formed of a single piece of tubing that can have a variable diameter, a variable wall thickness, and can provide a conduit for local drug delivery during treatment. The cylindrical body can include features such as spirals, slices, surface roughness, cage-like structures, struts, spines, coils, indentations and the like formed by laser cutting, mechanical machining, chemical machining, electro chemical machining, electrical discharge machining, or a combination thereof.

The transition portion and the proximal delivery portion can be include straight portion of tubing, a tubing with a spiral cut through the entire wall thickness, a tubing with spiral cut not through the entire wall thickness or a combination thereof and can be at least partially coated by biocompatible materials for lubricity.

The treatment portion can include peaks and valleys within its web-like structure to aid in clot retention and retrieval. Also, the treatment portion can include a marker wire within its inner lumen. The surface of the struts located in the treatment portion can include a coating or mechanically implemented roughness to enhance clot adhesion. The geometry of these struts can be different from a normal two dimensional configuration. The struts can twist and/or torque to form a more complicated geometry for better clot adhesion. One or more spiral grove and/or spiral volume can form along the length of the treatment portion. These volumes can house a clot during a procedure to prevent the clot from loosing or smaller portions breaking off.

Methods of removing a luminal occlusion from a lumen are also described comprising: removing substantially all of the luminal occlusion by entrapping the luminal occlusion in a thrombectomy device comprising a substantially cylindrical body formed from a single piece of tubing and including a proximal delivery portion, a transition portion, and a treatment portion, wherein the treatment portion includes a plurality of struts configured to encapsulate substantially all of the luminal occlusion.

In one embodiment, the luminal occlusion is a clot, an emboli, a thrombi, plaque, a cancerous growth, an excess tissue, a calcium deposit, or a combination thereof. In other embodiments, the luminal occlusion is in a blood vessel, a renal duct, a urethra, a fallopian tube, a vagina, an anus, intestines, a stomach, an esophagus, a bronchial tube, a lung, an ear canals, or a nostril.

Methods are also described of removing a luminal occlusion from a lumen comprising: inserting a thrombectomy device into the lumen to a location adjacent to the luminal occlusion, the thrombectomy device comprising a substantially cylindrical body formed from a single piece of tubing and including a proximal delivery portion, a transition portion, and a treatment portion, wherein the treatment portion a plurality of compressed struts forming a framework; expanding the compressed plurality of struts to encapsulate substantially all of the luminal occlusion; and removing the luminal occlusion by removing the thrombectomy device including the encapsulated luminal occlusion.

Kits are described including: a medical mechanical thrombectomy device comprising a substantially cylindrical body formed from a single piece of tubing and including a proximal delivery portion, a transition portion, and a treatment portion, wherein the treatment portion includes a plurality of struts forming a framework configured to encapsulate substantially all of the luminal occlusion; a catheter; and instructions for use.

In one embodiment, the kits further comprise a drug. The proximal delivery portion can include an inner lumen configured to deliver the drug to a treatment site. The catheter can be a microcatheter and the medical mechanical thrombectomy device can be pre-inserted into it or the mechanical thrombectomy device can be transferred into the microcathter from a transition tube or tubing during a procedure by a physician.

Also described are uses of a device for removing a luminal occlusion comprising: removing substantially all of the luminal occlusion by entrapping the luminal occlusion in a thrombectomy device comprising a substantially cylindrical body formed from a single piece of tubing and including a proximal delivery portion, a transition portion, and a treatment portion, wherein the treatment portion includes a plurality of struts configured to encapsulate substantially all of the luminal occlusion.

### BRIEF DESCRIPTION OF THE DRAWINGS

FIG. 1 illustrates an exemplary embodiment of a device as described herein.

FIG. 2 illustrates another view of an exemplary configuration of the device.

FIG. 3A-C illustrate views of an exemplary configuration of the device of FIG. 1. FIG. 3A is a perspective view. FIG. 3B illustrates a magnified view of the distal end of the treatment portion of the device. FIG. 3C illustrates a magnified view of the proximal end of the treatment portion of the device.

FIG. 4 illustrates a perspective view at an alternate angle of an exemplary configuration showing the spiral configuration of the major struts in the device. The view from this angle illustrates the spiral configuration of the major struts of the treatment portion.

FIG. 5 illustrates a laser cut pattern of an exemplary treatment portion.

FIG. 6 illustrates a laser cut pattern of an exemplary treatment portion design configuration with part of the transition portion.

FIG. 7 illustrates a laser cut pattern of an exemplary treatment portion structure design configuration.

FIG. 8A-C illustrate an exemplary process for using the described devices to remove a blood clot from a vessel.

FIG. 9A-C illustrate another exemplary process for using the described devices to remove a blood clot from a vessel.

FIG. 10A-C illustrate still another exemplary process for using the described devices to remove a blood clot from a vessel.

### DETAILED DESCRIPTION

Medical mechanical thrombectomy devices and methods of use for increasing blood flow through a blood vessel are described herein. In general, a device or device system includes an elongate member (proximal portion) and an expandable member (distal portion) fabricated from a single piece of material. The expandable member is configured to be inserted into a blood vessel and defines multiple spaces/openings in a wall of an expandable member located in a treatment portion. The expandable member generally has a compacted configuration for delivery and insertion into the target location of a blood vessel and an expanded configuration in which the expandable member defines an inner lumen to reestablish blood flow communication and to engage/receive embolus/clots with the multiple space/openings on it. The expandable member can include a first component having a stent like structure with multiple space/openings in its wall to help engage the embolus/clot and establish structural integrity of the device.

The present devices can overcome shortcomings of existing technologies. The present devices can be delivered to the target vasculature in a smooth fashion, can be retrieved safely, and/or can remove substantially all of an embolus. Substantially all of an embolus as used herein can mean a whole intact embolus. In other embodiments, substantially all can mean about 99%, about 98%, about 97%, about 96%, about 95%, about 94%, about 93%, about 92%, about 91%, or about 90% of the entire embolus. In still other embodiments, the device can remove about 85%, about 80%, about 75%, about 70%, at least about 70%, at least about 80%, at least about 90%, at least about 95%, at least about 97%, at least about 99%, between about 90% and about 99%, or between about 95% and about 100% of the entire embolus. In some embodiments, substantially all of an embolus is removed and smaller pieces are caught at the distal end of the treatment portion.

In use, the devices described can be compacted to a low profile, loaded onto a delivery system, and delivered to the target location in the vessel by a medical procedure such as by use of a delivery catheter, for example, a micro catheter. The devices can be released from the delivery system when the target implant site is reached and recovered to its normal expanded profile by elastic energy stored in the devices themselves (self-expandable device). In other embodiments, expansion can be from externally applied energy, such as but not limited to heat or ultrasound vibration.

Further, the devices described can be deployed at the site of the embolus or can be deployed distal to the embolus. For example, when dealing with a long embolus, a device can be used to remove the embolus from the proximal portion to distal with multiple passes, until substantially all of the embolus is removed. In other embodiments, the treatment portion can attached or intertwine itself into the distal end of an embolus and pull substantially all of the embolus out in a single pass.

Also, the present devices can have a seamless transition from delivery portion to treatment portion. In some embodiments, the devices can be fabricated from a single piece of biocompatible material tubing which exhibits super elastic or shape memory properties, e.g. nitinol. This feature can dramatically reduce an unintentional separation of the treatment device from a delivery wire. In addition, the transition portion can be fabricated from the same piece of tubing with variable flexibilities for optimal delivery and navigation characteristics.

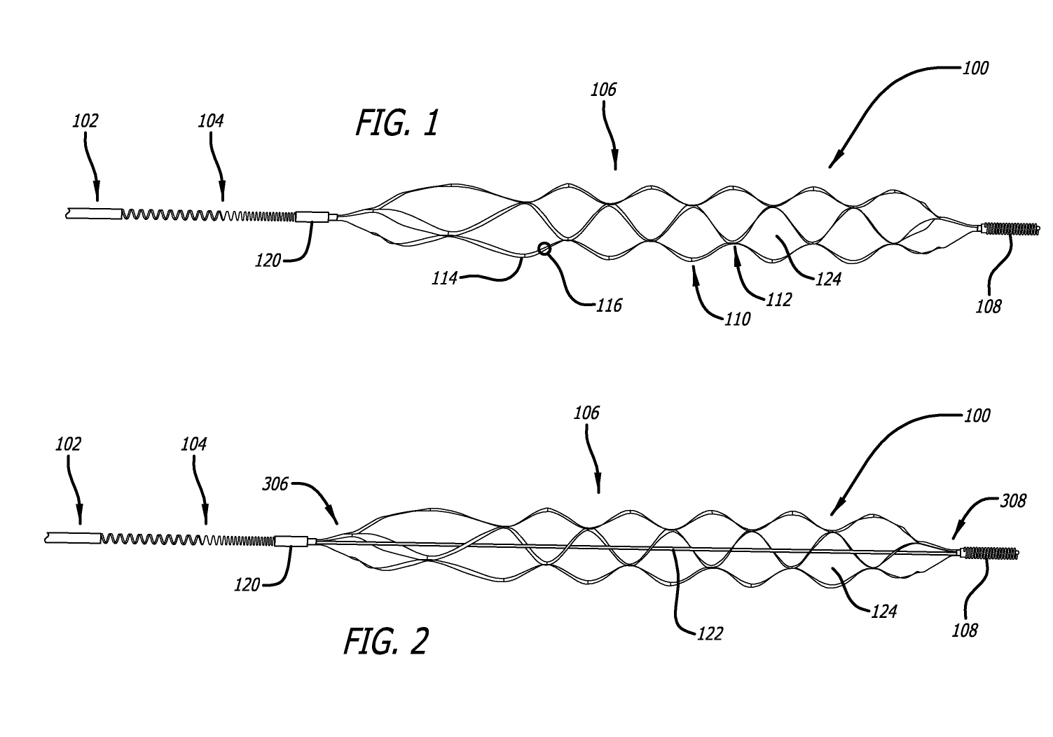

As illustrated in FIG. 1, a medical mechanical thrombectomy device 100 includes a proximal delivery portion 102, a transition portion 104, a treatment or capture portion 106, and a distal portion 108. Treatment portion 106 can contain peaks 110 and valleys 112 formed by the spaces/openings along its length. As such, the profile of treatment portion 106 may not be “smooth”. The major frame of peaks 110 and valleys 112 can be formed by two or more spines 114 in a helix/spiral configuration to form a strut 116. Struts used to form treatment portion can form a web-like, net, mesh, and/or framework to capture a luminal occlusion. In some embodiments, treatment portion can be formed of struts including a plurality of spines. The peaks 110, valleys 112, and spines 114 can help to improve the embolus affinity for better clot adhesion/affinity and retrieval during a procedure. Further, peaks 110 can be used to collect and retain the clot during use of the device. Valleys 112 can be used to store a clot volume and reduce mechanical force applied onto it (e.g., to prevent clot from breaking).

The devices can be made from one piece of nitinol super elastic material or nitinol shape memory alloy tubing. The device can also be made from other biocompatible materials that exhibit super elastic or shape memory properties. Laser cutting, mechanical machining, chemical machining, electrochemical machining, EDM, etc. or a combination thereof can be used to make a device as described herein.

Transition portion 104 including a variable flexibility (e.g., spiral cut through partial or entire wall thickness) can be built between proximal delivery portion 102 and treatment portion 106 to facilitate smooth device delivery and retrieval. Besides a spiral cut configuration of transition portion 104, other geometries can also be cut with an unlimited number of variations. In some embodiments, transition portion 104 can provide flexibility to the device. This flexibility can assist in allowing the devices to maneuver through tight corners in vessels during device delivery, device extraction, and/or a clot removal procedure.

The profile (diameter and wall thickness) of the single piece tubing material used to form the devices can vary. Tubing used to form the devices described herein can be straight tubing and/or can have variable wall thickness along the length. For example, the diameter at the proximal delivery portion 102 of the tubing can be smaller for easy delivery, but the diameter of the tubing in treatment portion 106 can be larger than the proximal delivery portion 102 (the tubing has variable diameter along the length). The larger diameter in the treatment portion 106 can allow different and/or more design configurations in that portion. Medical mechanical thrombectomy device 100 at its largest cross-section can have a diameter that allows deployment through a lumen or vessel. In aspects of this embodiment, at its largest cross-section, medical mechanical thrombectomy device 100 can have a diameter of less than about 10 mm, less than about 5 mm, less than about 4 mm, less than about 3 mm, less than about 2 mm, less than about 1 mm, less than about 0.5 mm, less than about 0.1 mm, less than about 0.05 mm, less than about 0.01 mm, less than about 0.005 mm, less than about 0.001 mm, between about 10 mm and about 1 mm, between about 5 mm and about 0.005 mm. between about 10 mm and about 0.001 mm, between about 1 mm and about 0.05 mm, or between about 2 mm and about 0.001 mm.

One or more struts 116 can be arranged in treatment portion 106 near the distal portion 108 to collect loose embolus during a procedure if any are present. The struts 116 in the stent like, web-like, net-like, framework, can be an expandable structure and can form an angle with the long axis of device 100 suitable to capture a luminal occlusion. In aspects of this embodiment, the angle can be between about 1 degree and about 179 degrees, between about 5 degrees and about 175 degrees, between about 10 degrees and about 170 degrees, between about 15 degrees and about 165 degrees, between about 20 degrees and about 160 degrees, about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, about 90 degrees, about 95 degrees, about 100 degrees, about 105 degrees, about 110 degrees, about 115 degrees, about 120 degrees, about 125 degrees, about 130 degrees, about 135 degrees, about 140 degrees, about 145 degrees, about 150 degrees, about 155 degrees, about 160 degrees, about 165 degrees, about 170 degrees, or about 175 degrees.

The framework formed by struts 116 can exhibit any hoop strength sufficient to keep the framework open during an extraction procedure. In another embodiment, the hoop strength can be sufficient to pin a clot between the framework and a vessel wall during extraction of the clot. Hoop strength can be between about 0.01 gf/mm and about 200 gf/mm, between about 0.05 gf/mm and about 150 gf/mm, between about 0.1 gf/mm and about 100 gf/mm, about 0.01 gf/mm, about 0.02 gf/mm, about 0.03 gf/mm, about 0.04 gf/mm, about 0.05 gf/mm, about 0.06 gf/mm, about 0.07 gf/mm, about 0.08 gf/mm, about 0.09 gf/mm, about 0.1 gf/mm, about 0.5 gf/mm, about 1 gf/mm, about 10 gf/mm, about 20 gf/mm, about 30 gf/mm, about 40 gf/mm, about 50 gf/mm, about 100 gf/mm, about 200 gf/mm, or about 300 gf/mm.

For example, for ischemic stroke treatment, the expandable framework of struts located in treatment portion 106 may be flexible enough to negotiate the torturous vasculature of the brain without severely modifying the vessel profile through the vasculature pathway, or at a target location. The compacted profile of the expandable stent-like member may be small enough to reach the target treatment site.

Struts 116 can be twisted along their longer axis for improved clot affinity and retention. The strut surface can be treated by a surface modification technique for improved clot retention and retrieval. The surface modification technique can include, but is not limited to, mechanical surface roughness modification, chemical etching, physical vapor deposition (PVD), chemical vapor deposition (CVD), surface coating, micro pinning, etc. In some embodiments, struts 116 can be mechanically, chemically, and/or electrochemically treated to form a rough and/or porous surface for better adhesion between the device and a biological tissue such as an embolus.

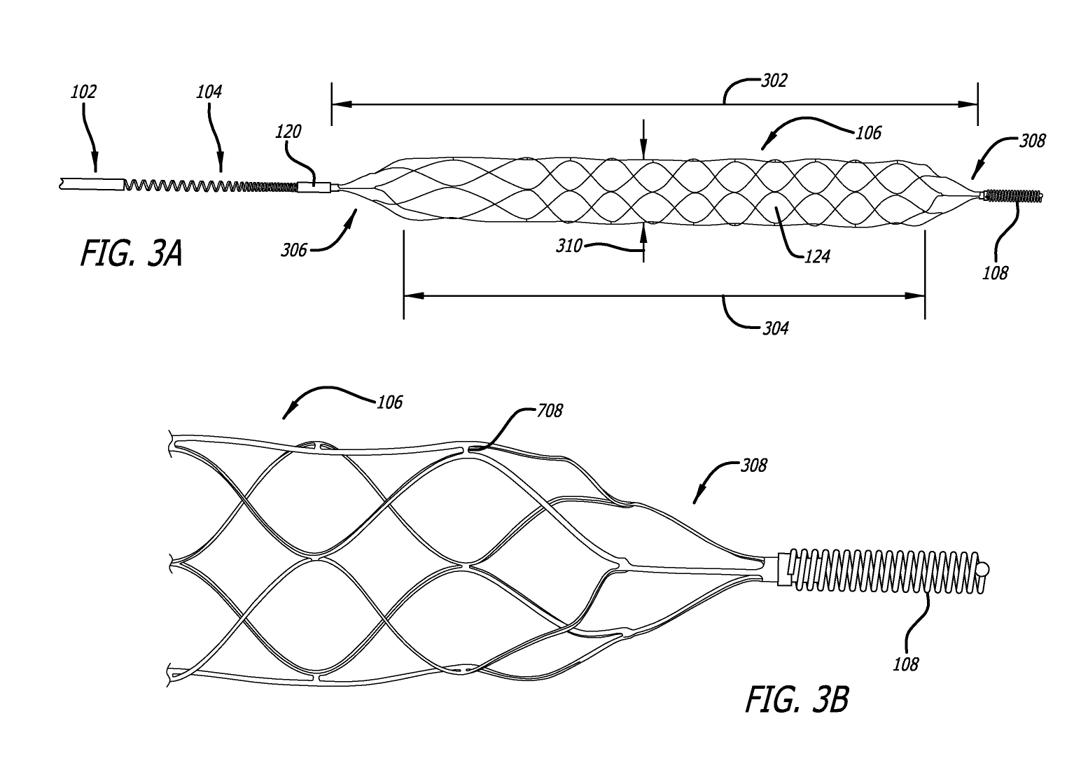

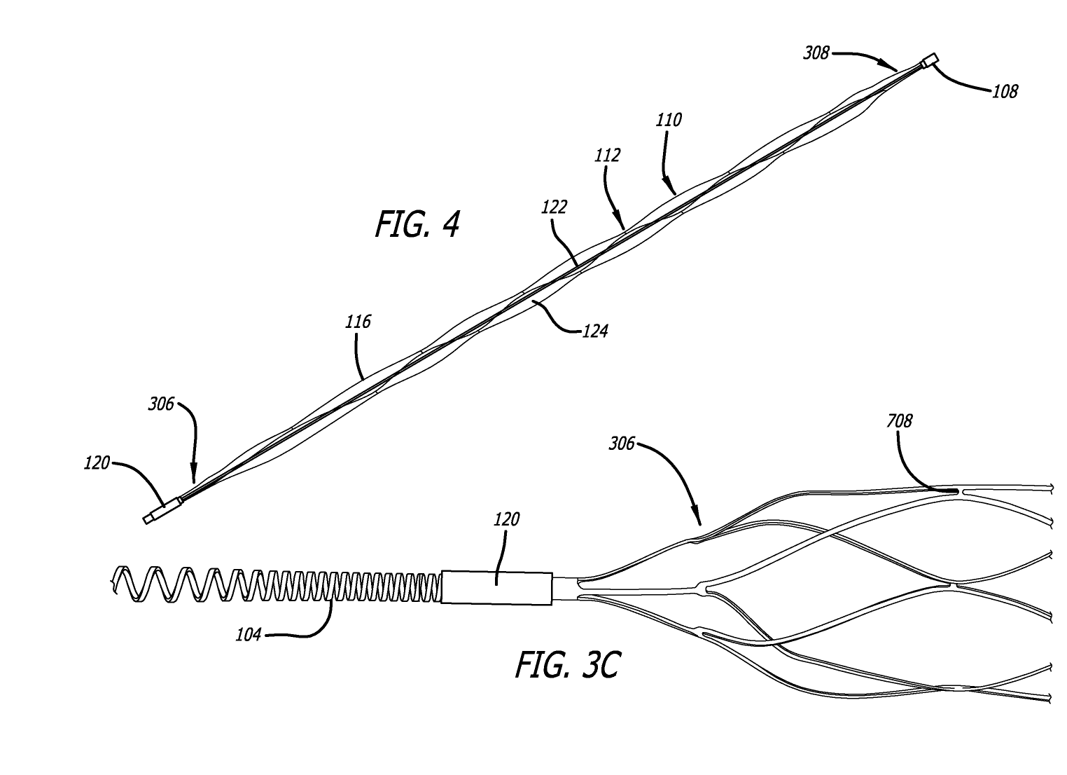

Treatment portion 106 can include tapered distal section 308 (see FIG. 3A) to collect small embolus break offs from a major clot(s) and to prevent it from migrating to a more distal area of the neurovasculature. A treatment portion can include, and may be referenced to include the combination of distal portion 108, treatment portion 106, marker band portion 120 and transition portion 104.

Medical mechanical thrombectomy device 100 can be made from either a metallic biocompatible material such as Nitinol, stainless steel, Co—Cr base alloy, Ta, Ti, etc., a polymer based biocompatible material such as polymers with shape memory effect, PTFE, HDPE, LDPE, Dacron, Polyester, etc., or a combination thereof.

Medical mechanical thrombectomy device 100 can be fully or partially coated. In some embodiments, different sections of the device can each be fully or partially coated. For example, treatment portion 106 can be fully or partially coated with polymers, polymer systems, one or more chemicals, or one or more drugs or other bioagents to prevent clotting and/or for the better adhesion between the device and embolus. The device surface can be treated to form different surface layers such as an oxidation layer, Nitro or carbonized or N-C-combined surface layer, etc. for better adhesion between device and embolus.

FIG. 2 illustrates another design configuration of medical mechanical thrombectomy device 100. Compared with the configuration illustrated in FIG. 1, marker wire or coil 122 can be placed in lumen 124 of treatment portion 106 through its partial or entire length for improved visibility during a procedure. Marker wire or coil 122 can help with clot retention and retrieval. The marker wire surface can be treated and/or modified for improved clot retention and retrieval properties. The marker wire can also be formed in a spiral or coiled configuration. Surface treatment and/or modification techniques can include, but are not limited to, mechanical surface roughness modification, chemical etching, physical vapor deposition (PVD), chemical vapor deposition (CVD), surface coating, micro pinning, etc.

Medical mechanical thrombectomy device 100 can include one or more radiopaque markers to help position the device using standard imaging equipment. A radiopaque marker can be formed of or include a material that can be visualized using standard fluoroscopy or other medical imaging techniques and/or equipment. In some embodiments, radiopaque material can include a marker coil, a marker band, a marker wire, a marker coating, a polymer extrusion blended with one or more radiopaque materials, etc., or a combination thereof. Radiopaque materials can include Pt, Pt—Ir alloy, W, Ta, Au, iodine based contrast agents, Ba, Ga, microbubble contrast agents, CO₂, Th, and other typical radiopaque materials or polymer loaded with radiopaque materials. Further, the radiopqaque marker can be located at any convenient location on a device. In some embodiments, the radiopaque marker can be located on at least parts of the distal portion 108, the proximal delivery portion 102, or either partially or entirely through the entire inner lumen of treatment portion 106. For example, medical mechanical thrombectomy device 100 can include a marker coil at distal portion 108 at the distal end of treatment portion 106, marker band portion 120 at the proximal end of treatment portion 106, or a marker wire 122 within lumen 124. In one embodiment, bands and coils can be used interchangeably.

Radiopaque markers can also be attached on any other portion of medical mechanical thrombectomy device 100. One way to gain the full visibility can be to run a radiopaque material through the entire or partial lumen of a delivery wire. Markers can also be placed on the treatment portion and/or struts 116 to aid in positioning.

Transition portion 104 can be seamless requiring no joints or bonding. Also, transition portion 104 can be modified with a number of variations to vary flexibility by having straight tubing, spiral cut through the wall thickness, or spiral cut partially through the wall thickness. When spiral cut, flexibility can be varied through variable pitch sizes across the length. In some embodiments, transition portion 104 can be covered by polymer tubing, polymer layering, polymer covering, polymer coating, or a combination thereof for optimization of deliverability and/or surface smoothness.

Medical mechanical thrombectomy device 100 can include an inner lumen axially traversing the length of the device running throughout proximal delivery portion. Such an inner lumen can be used for local drug delivery in the vasculature if needed and/or required. In one embodiment, the inner lumen travels along proximal delivery portion 102 and transition portion 104 can be used to deliver one or more drugs to the specific local area in the vessel being treated. The one or more medicine can include, but are not limited to anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, tPA, heparin, and transforming nucleic acids.

FIG. 3 illustrates a perspective view of medical mechanical thrombectomy device 100 including various measurements. Proximal delivery portion 102 can have a diameter between about 0.100 mm and about 2 mm, between about 0.120 mm and about 1.75 mm, between about 0.150 mm and about 0.125 mm, between about 0.127 mm and about 1.524 mm, about 0.100 mm, about 0.120 mm, about 0.125 mm, about 0.127 mm, about 0.130 mm, about 0.150 mm, about 0.170 mm, about 0.120 mm, about 0.20 mm, about 0.30 mm, about 0.40 mm, about 0.50 mm, about 0.60 mm, about 0.70 mm, about 0.80 mm, about 0.90 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm. The wall thickness of proximal delivery portion 102, if tubular, can be between about 20 μm and about 200 μm, between about 40 μm and about 100 μm, between about 20 μm and about 100 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm. The length of proximal delivery portion 102 can be between about 60 cm and about 200 cm, between about 80 cm and about 100 cm, between about 60 cm and about 100 cm, about 60 cm, about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, or about 200 cm.

Transition portion 104 can have a diameter between about 0.100 mm and about 2 mm, between about 0.120 mm and about 1.75 mm, between about 0.150 mm and about 0.125 mm, between about 0.127 mm and about 1.524 mm, about 0.100 mm, about 0.120 mm, about 0.125 mm, about 0.127 mm, about 0.130 mm, about 0.150 mm, about 0.170 mm, about 0.120 mm, about 0.20 mm, about 0.30 mm, about 0.40 mm, about 0.50 mm, about 0.60 mm, about 0.70 mm, about 0.80 mm, about 0.90 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm. The pitch size and diameter of transition portion 104 can vary along the length for varying flexibilities. Further, transition portion 104 can be covered by a polymer layer or tubing for optimal deliverability. The spiral cut can either be through the entire wall thickness of the tubing or only partially through the wall thickness leaving a groove on the surface. In the case wherein the spiral cut is through the entire wall thickness, transition portion 104 can have a real spiral profile. The length of transition portion 104 can be between about 10 mm and about 150 cm, between about 50 mm and about 100 cm, between about 100 mm and about 50 cm, about 10 mm, about 20 mm, about 30 mm, about 40 mm, about 50 mm, about 60 mm, about 70 mm, about 80 mm, about 90 mm, about 100 mm, about 200 mm, about 300 mm, about 400 mm, about 500 mm, about 600 mm, about 700 mm, about 800 mm, about 900 mm, about 100 cm, about 110 cm, about 120 cm, about 130 cm, about 140 cm, or about 150 cm.

The length of marker band portion 120 can be between about 0.5 mm to about 10 mm, between about 1 mm and about 5 mm, between 0.5 mm and about 5 mm, between about 5 mm and about 10 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 3 mm, about 4 mm, about 5 mm, about 6 mm, about 7 mm, about 8 mm, about 9 mm, or about 10 mm. Marker band portion 120 can have a diameter between about 0.100 mm and about 5 mm, between about 0.120 mm and about 1.75 mm, between about 0.150 mm and about 0.125 mm, between about 0.127 mm and about 1.524 mm, about 0.100 mm, about 0.120 mm, about 0.125 mm, about 0.127 mm, about 0.130 mm, about 0.150 mm, about 0.170 mm, about 0.120 mm, about 0.20 mm, about 0.30 mm, about 0.40 mm, about 0.50 mm, about 0.60 mm, about 0.70 mm, about 0.80 mm, about 0.90 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3 mm, about 3.5 mm, about 4 mm, about 4.5 mm, or about 5 mm.

Treatment portion 106 can have two lengths which include a total length 302 and effective length 304. Total length 302 can be between about 8 mm and about 80 mm, between about 10 mm and about 60 mm, between about 8 mm and about 50 mm, between about 20 mm and about 80 mm, between about 20 mm and about 60 mm, about 8 mm, about 9 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, or about 80 mm. Effective length 304 can be between about 5 mm and about 60 mm, between about 10 mm and about 40 mm, between about 8 mm and about 20 mm, between about 20 mm and about 60 mm, between about 20 mm and about 40 mm, about 5 mm, about 6 mm, about 7 mm, about 8 mm, about 9 mm, about 10 mm, about 15 mm, about 20 mm, about 25 mm, about 30 mm, about 35 mm, about 40 mm, about 45 mm, about 50 mm, about 55 mm, or about 60 mm. Effective length 304 can be defined as an area having uniform diameter and capable of engaging a luminal occlusion.

First end 306 and second end 308 of treatment portion 106 can be tapered to achieve a smooth transition during delivery and retrieval. The diameter 310 of treatment portion 106 can be between about 1.5 mm and about 12 mm, between about 2 mm and about 10 mm, between about 5 mm and about 10 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, or about 12 mm.

Distal portion 108 can have a length between about 0.5 mm to about 100 mm, between about 1 mm and about 50 mm, between 0.5 mm and about 25 mm, between about 5 mm and about 50 mm, about 0.5 mm, about 0.6 mm, about 0.7 mm, about 0.8 mm, about 0.9 mm, about 1 mm, about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm or about 100 mm. Distal portion 108 can have a diameter between about 0.100 mm and about 10 mm, between about 0.120 mm and about 1.75 mm, between about 0.150 mm and about 0.125 mm, between about 0.127 mm and about 1.524 mm, about 0.100 mm, about 0.120 mm, about 0.125 mm, about 0.127 mm, about 0.130 mm, about 0.150 mm, about 0.170 mm, about 0.120 mm, about 0.20 mm, about 0.30 mm, about 0.40 mm, about 0.50 mm, about 0.60 mm, about 0.70 mm, about 0.80 mm, about 0.90 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3 mm, about 3.5 mm, about 4 mm, about 4.5 mm, or about 5 mm. In some embodiments, distal portion 108 can include struts to collect loose embolus during use.

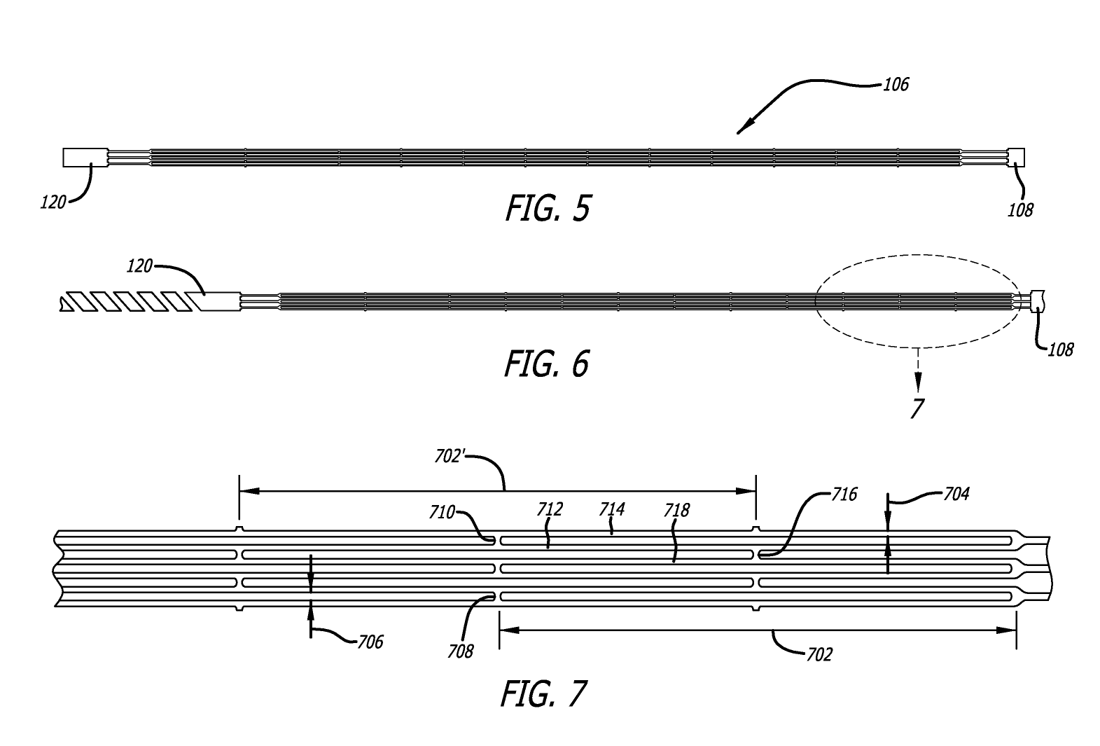

FIGS. 5 and 6 illustrate a magnified view of a laser cutting pattern of treatment portion 106. Treatment portion 106 can be laser cut from the same tubing used to form marker band portion 120 mounting location and transition portion 104, and then expanded to its final expanded shape/profile through a heat setting process. In one embodiment, when a single piece of tubing is used to form medical mechanical thrombectomy device 100, tubing with a tapered profile (variable diameter) can be used. Treatment portion 106 can be laser cut from the tubing portion which has the same diameter as its expanded diameter and no shape setting is needed. Treatment portion 106 can be either microblasted or electropolished after shape setting. In another embodiment, treatment portion 106 can be electropolished and then microblasted after shape setting. The final surface of treatment portion 106 can be modified/performed afterward. In one embodiment, the final surface of treatment portion 106 can be a microblasted surface including a mechanical roughness.

FIG. 7 illustrates an even more magnified view of the treatment portion of FIGS. 5 and 6. Struts 116 can include one or more unit cells 702. The length of a unit cell within treatment portion 106 can be between about 1 mm and about 40 mm, between about 5 mm and about 30 mm, between about 1 mm and about 20 mm, between about 20 mm and about 40 mm, about 1 mm, about 2 mm, about 3 mm, about 4 mm, about 5 mm, about 10 mm, about 15 mm, about 20 mm, about 25 mm, about 30 mm, about 35 mm, or about 40 mm. Struts 116 can have a width 704 of between about 10 μm and about 300 μm, between about 20 μm and about 200 μm, between about 50 μm and about 100 μm, between about 10 μm and about 100 μm, about 10 μm, about 20 μm, about 30 μm, about 40 μm, about 50 μm, about 60 μm, about 70 μm, about 80 μm, about 90 μm, about 100 μm, about 150 μm, about 200 μm, about 250 μm, or about 300 μm. An interstrut space 706 can be between about 0.25 μm and about 3 mm, between about 0.5 μm and about 100 μm, between about 0.25 μm and about 1 mm, about 0.25 μm, about 0.50 μm, about 0.75 μm, about 1 μm, about 10 μm, about 50 μm, about 100 μm, about 500 μm, about 1 mm μm, about 2 mm, or about 3 mm. Further, each strut can be held to an adjacent strut by strut bridge 708. Strut bridges can alternate on opposite sides of a strut. For example, in FIG. 7, first bridge 710 connects first strut 712 to second strut 714. Likewise, second bridge 716 connects first strut 712 to third strut 718. In another embodiment, bridges can be located at each connection position; in other words, bridges may not need to alternate.

The devices can include portions that have been surface treated. The surface treatments can improve performance of the various portions of the device. Marker band portion 120 and transition portion 104 can either be coated or covered either entirely or partially by typical biocompatible materials for lubricity. The surface of treatment portion 106 can include either a positive or negative charge for improved clot adhesion. This positive or negative charge can be in the form of a charged polymer applied to at least a portion of treatment portion 106. The surface of treatment portion 106 can also be either mechanically or chemically treated to have a rough surface for improved clot adhesion. The rough surface can be achieved by application of a porous surface coating or layer, microblasting or micropinning the surface, or using an irregular strut geometry/arrangement, for example, twisted struts and/or struts with different angles.

When using a device as described herein, treatment portion 106 can be compacted to a smaller delivery profile than its expanded dimensions and loaded into a delivery catheter. In some embodiments, the devices can be loaded into a microcatheter. In other embodiments, the microcatheter can be of a size 0.5 Fr, 1 Fr, 2 Fr, 3 Fr, 4 Fr, 5 Fr, 6 Fr, 7 Fr, 8 Fr, 9 Fr, 10 Fr, 11 Fr, or 12 Fr. After loading a compacted device into a catheter or microcatheter, the entire system can be delivered to a target location in a vessel to retrieve a clot.

The present devices can be used to capture a luminal occlusion such as a vessel occlusion or other vessel blockage. In some embodiments, the devices can be used to capture any appropriate obstruction in virtually any lumen. Occlusions can include, clots, emboli, thrombi, plaque, cancerous growths, excess tissues, calcium, kidney stones, wax, cerumen, and the like. Appropriate lumen can include neurovasculature, arteries, veins, renal ducts, urethra, fallopian tubes, vagina, anus, intestines, stomach, esophagus, bronchial tubes, lungs, ear canals, nostrils and the like.

Likewise the devices can be used to treat or cure medical conditions such as, but not limited to thrombosis, stroke, heart attack, cancer, athlerosclerosis, ateriosclerosis, bowel obstruction, renal calculus, and the like.

Kits including the described devices can comprise a medical mechanical thrombectomy device, a catheter or microcatheter, and instructions for use. In other embodiments, the kits can further include a drug for local delivery at a treatment site. In some embodiments, the kits can include a separate medical mechanical thrombectomy device, transfer tubing and a catheter or microcatheter. In other embodiments, the kits can include a medical mechanical thrombectomy device pre-inserted into transfer tubing or other lumen allowing the device to be loaded into a micorcatheter during a procedure.

To use a device as described herein, as a first step, a guidewire is inserted in the vasculature until reachs the target treatment site; a microcatheter is advanced over the guidewire until it reached the targeted treatment site; the guidewire is removed, and leave the microcatheter in place; the mechanical thrombectomy device is then inserted into the microcatheter from its proximal end, and advanced to the treatment site though the inner lumen of the microcatheter. Or a medical mechanical thrombectomy device is pre-loaded and fitted into a micocatheter. In other embodiments, a device can be prepackaged in a catheter or microcatheter and is ready for deploying out of the package. For example, to remove a blood clot from the brain, the microcatheter is instered into an appropriate artery or vein (e.g., the femoral artery) and guided using an appropriate imaging technique (e.g., fluoroscopy) to the desired treatment site where treatment is to commence. Other vessels can be used, the above is simple an example of a procedure than may be used.

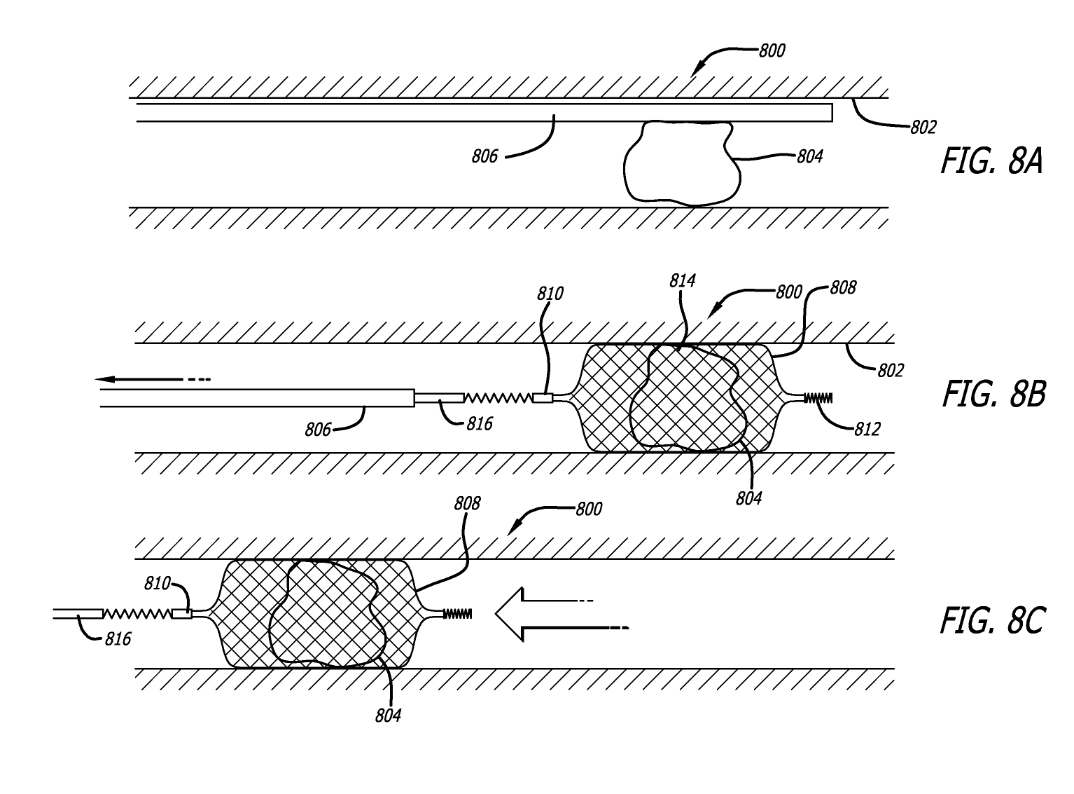

As illustrated in FIGS. 8A-C, treatment site 800 includes a vessel 802 obstructed by a luminal occlusion such as a blood clot 804. Microcather 806 is maneuvered to a location traversing blood clot 804 by the aid of a guidewire (not shown). Then, microcatheter 806 is partially removed to reveal medical mechanical thrombectomy device 816. By removing microcather 806, treatment portion 808 is self expanded thereby entraps blood clot 804 within struts 814 of treatment portion 808. After expansion and entrapment, the entire system including microcatheter 806 and blood clot 804 is removed from vessel 802. Imaging of first marker 810 and second marker 812 can aid in placement of treatment portion 808. In one embodiment, substantially all of blood clot 804 is removed from the vessel. In another embodiment, all of blood clot 804 is removed from the vessel. In one embodiment, negative pressure may be applied during clot retrieval/removal sucking blood clot 804 into the microcatheter 806 thereby removing it.

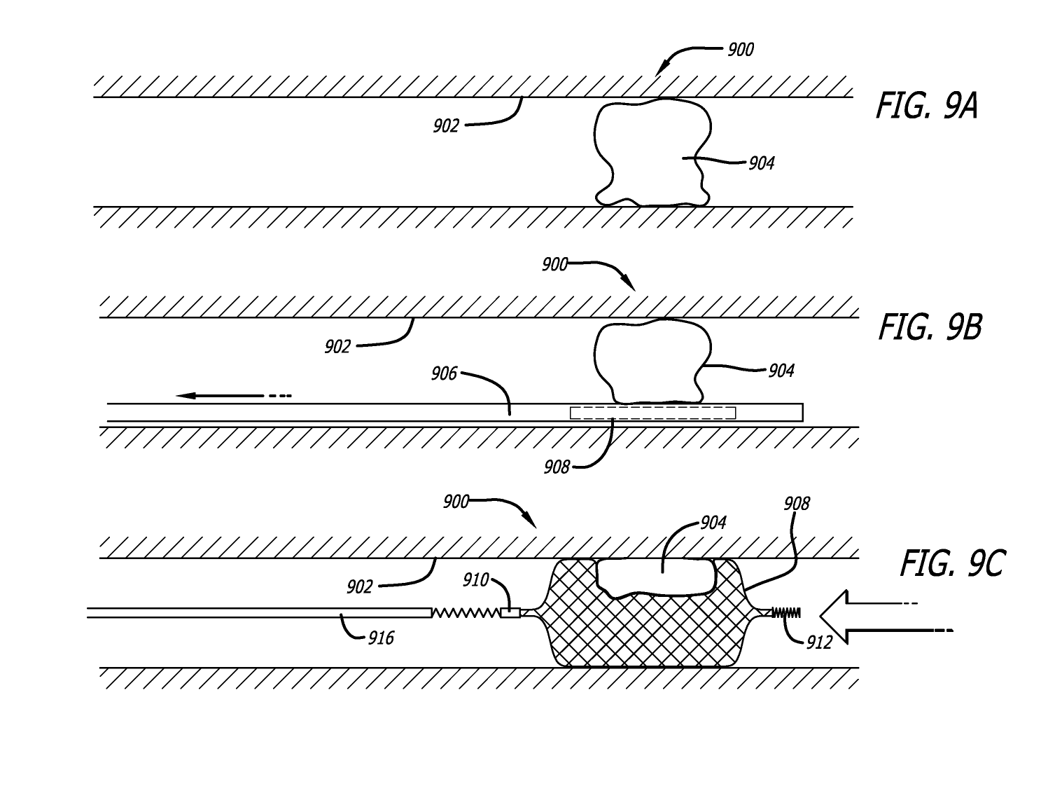

As illustrated in FIGS. 9A-C, treatment site 900 includes a vessel 902 obstructed by a luminal occlusion such as a blood clot 904. Microcather 906 is maneuvered to a location traversing blood clot 804 by the aid of a guidewire (not shown). Then, microcatheter 906 is partially removed to reveal medical mechanical thrombectomy device 916. By removing microcather 906, treatment portion 908 is self expanded thereby trapping blood clot 904 between struts 914 of treatment portion 908 and the vessel wall. After expansion and trapping of blood clot 904, the entire system including microcatheter 906 and blood clot 904 is removed from vessel 902. Imaging of first marker 910 and second marker 912 can aid in placement of treatment portion 908. In one embodiment, substantially all of blood clot 904 is removed from the vessel. In another embodiment, all of blood clot 904 is removed from the vessel. In one embodiment, negative pressure may be applied during clot retrieval/removal sucking blood clot 904 into microcatheter 906 thereby removing it.

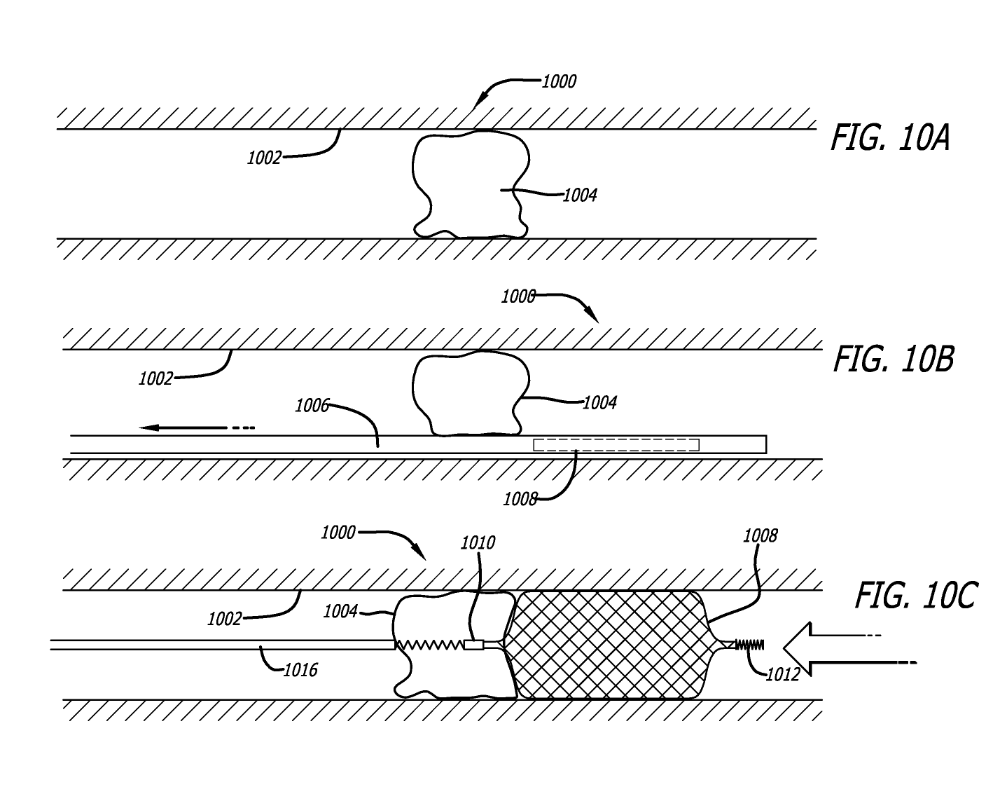

As illustrated in FIGS. 10A-C, treatment site 1000 includes a vessel 1002 obstructed by a luminal occlusion such as a blood clot 1004. Microcather 1006 is maneuvered past blood clot 804 by the aid of a guidewire (not shown) to a location where treatment portion 1008 can expand distally in relation to blood clot 1004. Then, microcatheter 1006 is partially removed to reveal medical mechanical thrombectomy device 1016. By removing microcather 1006, treatment portion 1008 is self expanded thereby traversing the diameter of vessel 1002 yet still allowing blood to flow through it. Then, the entire system including microcatheter 1006 and medical mechanical thrombectomy device 1016 is extracted thereby engaging the proximal portion of treatment portion 1008 to engage and pull out blood clot 1004. Imaging of first marker 1010 and second marker 1012 can aid in placement of treatment portion 1008 and removal of blood clot 1004. In one embodiment, substantially all of blood clot 1004 is removed from the vessel. In another embodiment, all of blood clot 1004 is removed from the vessel. In one embodiment, negative pressure may be applied during clot retrieval/removal sucking blood clot 1004 into microcatheter 1006 thereby removing it.

Although detailed descriptions of the invention are disclosed herein, it needs to be understood that the disclosed descriptions are merely exemplary of the invention that may be embodied in various and alternative forms based on the basic idea or design principal disclosed. Specific structural and functional details disclosed herein are not to be interpreted as limiting, but merely as a basis for teaching skilled ones in the art to variously employ the vasculature mechanical thrombectomy device embodiments.

It will be appreciated by those skilled in the art that changes could be made to the example embodiments described in this invention without departing from the broad invention concept/idea thereof. While particular embodiments of the present invention have been described, it is not intended to limit the invention only to any specific embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A thrombectomy device comprising:
a substantially cylindrical body formed from a single piece of tubing and including a proximal delivery portion, a transition portion, and an expandable treatment portion,
wherein the expandable treatment portion includes one or more struts configured to engage, capture, or encapsulate substantially all of a luminal occlusion.

2. The thrombectomy device of claim 1, wherein the single piece of tubing is formed of a nitinol super elastic material or nitinol shape memory alloy.

3. The thrombectomy device of claim 1, wherein the substantially cylindrical body is formed by laser cutting, mechanical machining, chemical machining, electro chemical machining, electrical discharge machining, or a combination thereof.

4. The thrombectomy device of claim 1, wherein the substantially cylindrical body has a variable diameter.

5. The thrombectomy device of claim 1, wherein the substantially cylindrical body has variable wall thickness.

6. The thrombectomy device of claim 1, wherein the substantially cylindrical body includes an inner lumen configured for local drug delivery.

7. The thrombectomy device of claim 1, further comprising at least one radiopaque element.

8. The thrombectomy device of claim 7, wherein the at least one radiopaque element is a marker band or a marker coil.

9. The thrombectomy device of claim 1, wherein the transition portion is a straight piece of tubing, a tubing with a spiral cut through the entire wall thickness, a tubing with spiral cut not through the entire wall thickness, or a combination thereof.

10. The thrombectomy device of claim 1, wherein the proximal delivery portion and the transition portion are each independently at least partially coated by biocompatible materials for lubricity.

11. The thrombectomy device of claim 1, wherein the proximal delivery portion is a straight piece of tubing having a variable diameter, a variable wall thickness, or a combination thereof.

12. The thrombectomy device of claim 1, wherein the expandable treatment portion is coated with a polymer having positive charge or a negative charge for improved clot adhesion.

13. The thrombectomy device of claim 1, wherein the treatment portion is mechanically or chemically treated to have a rough surface for improved clot adhesion.

14. The thrombectomy device of claim 13, wherein the rough surface is a porous surface coating, a porous surface layer, a microblasted surface, a micropinning, an irregularly configured strut geometry, or a combination thereof.

15. The thrombectomy device of claim 1, wherein the treatment portion can have peaks and valleys for clot retention and retrieval.

16. The thrombectomy device of claim 1, wherein the struts form spiral configuration along the treatment portion to facilitate device delivery and clot retention and retrieval.

17. The thrombectomy device of claim 1, wherein the struts are twisted for clot interaction.

18. The thrombectomy device of claim 1, further comprising a marker wire within the inner lumen.

19-75. (canceled)
